(19) United States
(12) Patent Application Publication
(10) Pub. No.: US 2024/0368221 A1
(43) Pub. Date: Nov. 7, 2024

(54) IMPATIENS BALSAMINA-DERIVED ANTI-BACTERIAL PEPTIDE AND ANTI-BACTERIAL COMPOSITION CONTAINING SAME

(71) Applicant: V&CO CO., LTD., Seoul (KR)

(72) Inventors: Sang Do CHA, Incheon (KR); Min Seok CHO, Suwon-si (KR); Won Seok CHOI, Daegu (KR); Jang Hee CHO, Incheon (KR); Ji Won YU, Seoul (KR)

(73) Assignee: V&CO CO., LTD., Seoul (KR)

(21) Appl. No.: 18/686,124

(22) PCT Filed: Oct. 15, 2021
(86) PCT No.: PCT/KR2021/014309

§ 371 Date: Feb. 23, 2024

(30) Foreign Application Priority Data
Aug. 23, 2021 (KR) 10-2021-0110623

(51) Int. Cl. C07K 7/06; A23K 20/147; A61K 8/64; A61K 38/00; A61P 31/04; A61P 31/10

(52) U.S. Cl. CPC C07K 7/06; A23K 20/147; A61K 8/64; A61P 31/04; A61P 31/10; A61K 38/00

(57) ABSTRACT

The present invention relates to an Impatiens balsamina-derived anti-bacterial peptide and an anti-bacterial composition comprising same. Specifically, six types of peptide derivatives were prepared on the basis of the sequence of a conventional Impatiens balsamina-derived peptide Ib-AMP4 known to have anti-fungal activity, and a peptide derivative exhibiting anti-bacterial and anti fungal activity better than that of Ib-AMP4 and having weak cytotoxicity and hemolytic properties has been identified, and thus the peptide derivative, which is an Impatiens balsamina-derived anti-bacterial peptide, can be used, in the fields of medicine, cosmetics and food, as an anti-bacterial or anti-fungal pharmaceutical composition, antibiotic, food preservative, cosmetic preservative, pharmaceutical preservative or feed additive preservative, and the like.

### TECHNICAL FIELD

The present invention relates to an Impatiens balsamina-derived antimicrobial peptide and an antimicrobial composition containing the same, and more particularly, to an antimicrobial peptide that is derived from Impatiens balsamina, which exhibits antimicrobial and antifungal activity and has insignificant cytotoxic and hemolytic activity, and an antimicrobial or antifungal composition containing the same.

### BACKGROUND ART

Antibiotics with antimicrobial or antifungal effects are materials with selective toxicity, which kill microorganisms but have low toxicity to humans or animals and are not inactivated by enzymes in the body. Antibiotics become effective through a mechanism of inhibiting the proliferation of microorganisms by mainly suppressing DNA replication, the transcription and translation of genetic information, transport of transcription energy, and biosynthesis of the cell wall.

However, due to widespread and indiscriminate use of antibiotics, antibiotic-resistant strains are increasing. According to the antimicrobial resistance (AMR) hospital antibiotic report of the U.S. Centers for Disease Control and Prevention (CDC), approximately 100,000 people were infected with methicillin-resistant Staphylococcus aureus (MRSA) in 2006 due to misuse of antibiotics, and approximately 20,000 of them died. In 2010, in Korea, carbapenem-resistant enterobacteriaceae (CRE) containing the New Delhi metalo-beta-lactamase (NDM-1) gene referred to as super bacteria were found, indicating that the risk of resistant bacteria is increasing. Currently, most worldwide research on infectious diseases is focused on the development of antibiotics that selectively inhibit or control the metabolic or biosynthetic pathways of pathogens and the development of vaccines caused by culturing attenuated pathogens. The use of existing antibiotics is limited because they often exhibit side effects and toxicity by simultaneously inhibiting enzymes for regulating host's metabolic and biosynthetic pathways and have antibiotic resistance. There is also the problem that it is not possible to quickly respond to new pathogens through vaccine development alone. Therefore, there is an urgent need to develop a new paradigm of injection treatment, which can effectively control infectious diseases in the early stage, and recently, domestic and foreign researchers are focusing on antimicrobial peptides (AMPs) as a way to solve existing problems.

AMPs are components of the immediate non-specific defense mechanism of almost all species in the infection process. They are mainly positively charged (+2 to +9) and include 30% or more hydrophobic amino acid residues. Due to such characteristics, when an AMP comes into contact with the negatively-charged bacterial cell wall, an amphipathic α-helix or β-sheet may be formed and introduced into the cell membrane. When an AMP enters the cell membrane of a target bacterium, it is known that the positively-charged AMP binds to the negatively-charged bacterial, viral or fungal lipid membrane to cause the lipid membrane to destabilize and the potential of the cell membrane to disappear, resulting in the death of the microorganism. Natural-occurring AMPs are emerging as candidates for new antibiotics. Since these AMPs exhibit antimicrobial activity through different action mechanisms from compound-type antibiotics used conventionally, they are expected to solve the problems of antibiotic-resistant strains.

As an example, 4 types of closely related AMPs were isolated from the seeds of Impatiens balsamina and named Ib-AMP1, Ib-AMP2, Ib-AMP3, and Ib-AMP4, respectively. These peptides are basic and include 4 cysteine residues that form disulfide bonds between two molecules. Particularly, Ib-AMP4 has major advantages of inhibiting various phytopathogenic fungi such as Fusarium culmorum, Botrytis cinerea, and Mycosphaerella fijiensis and having no cytotoxicity to human cells. However, Ib-AMP4 appears to have low antimicrobial activity against gram-negative bacteria such as E. coli and has the disadvantage of being difficult to synthesize due to forming a disulfide bond and producing an undesired isomer and is expensive.

Therefore, as a result of attempting to develop Impatiens balsamina-derived AMPs that exhibit excellent antibiotic and antifungal activity and are easily synthesized, 3D structural analysis was performed based on the sequence of Impatiens balsamina-derived AMP Ib-AMP4 to prepare six types of peptide derivatives, and among these, peptide derivatives that have significantly excellent antimicrobial and antifungal activity and insignificant cytotoxic and hemolytic activity compared to Ib-AMP4 were identified. Thus, the present invention was completed.

### RELATED ART DOCUMENTS

(Patent Document 1) Korean Laid-open Publication No. 10-2020-0056194
(Non-Patent Document 1) Tailor R H, Acland D P, Attenborough S, et al. A novel family of small cysteine-rich AMPs from seed of Impatiens balsamina is derived from a single precursor protein. J Biol Chem. 1997, 272:24480-24487.
(Non-Patent Document 2) Thevissen K, Francois Isabelle E J A, Sijtsma L, et al. Antifungal activity of synthetic peptides derived from Impatiens balsamina AMPs Ib-AMP1 and Ib-AMP4. Peptides. 2005, 26:1113-1119.
(Non-Patent Document 3) Florez-Castillo J. M, Perullini M, Jobbagy M. et al. Enhancing Antibacterial Activity Against Escherichia coli K-12 of Peptide Ib-AMP4 with Synthetic Analogues. Int J Pept Res Ther. 2014, 20:365-369.

### Disclosure

### Technical Problem

Existing Impatiens balsamina-derived antimicrobial peptides (AMPs) exhibit antifungal activity, but have the disadvantage of low antimicrobial activity against gram-negative bacteria such as E. coli.

The present invention is directed to providing an Impatiens balsamina-derived peptide, such as an AMP that exhibits excellent antimicrobial and antifungal activity and has insignificant cytotoxic and hemolytic activity, and an antimicrobial or antifungal composition containing the same.

### Technical Solution

One aspect of the present invention provides an Impatiens balsamina-derived peptide consisting of the amino acid sequence of General Formula 1:

(N-terminus)-GPX¹RRYX²RR-(C-terminus) [General Formula 1]

In the above formula, X¹ is any one selected from the group consisting of glycine (G) and tryptophane (W), and X² is any one selected from the group consisting of cysteine (C), alanine (A), and tryptophane (W).

Another aspect of the present invention provides an antimicrobial or antifungal pharmaceutical composition that includes the Impatiens balsamina-derived peptide as an active ingredient.

Still another aspect of the present invention provides an antibiotic that includes the Impatiens balsamina-derived peptide as an active ingredient.

Yet another aspect of the present invention provides a food preservative that includes the Impatiens balsamina-derived peptide as an active ingredient.

Yet another aspect of the present invention provides a cosmetic preservative that includes the Impatiens balsamina-derived peptide as an active ingredient.

Yet another aspect of the present invention provides a pharmaceutical preservative that includes the Impatiens balsamina-derived peptide as an active ingredient.

Yet another aspect of the present invention provides a feed additive that includes the Impatiens balsamina-derived peptide as an active ingredient.

### Advantageous Effects

In the present invention, six types of peptide derivatives were prepared by performing 3D structural analysis based on the conventional sequence of Impatiens balsamina-derived peptide Ib-AMP4 with known antifungal activity, and among these, peptide derivatives that have significantly excellent antimicrobial and antifungal activity and insignificant cytotoxic and hemolytic activity, compared to Ib-AMP4, were identified. The peptide derivatives are Impatiens balsamina-derived antimicrobial peptides, which can be used as an antimicrobial or antifungal pharmaceutical composition, an antibiotic, a food preservative, a cosmetic preservative, a pharmaceutical preservative, or a feed additive, which is used in the pharmaceutical, cosmetic, and food industries.

### DESCRIPTION OF DRAWINGS

FIG. 1 shows the 3D structures of six types of peptide derivatives, such as VESCA-NH2, VESCA-NH4, VESCA1, VESCA2, VESCA3, and VESCA-OH, selected based on Impatiens balsamina-derived AMP, Ib-AMP4, and the sequence thereof.

FIG. 2 shows the cell viability of the bee venom-derived AMP, melittin, and the Impatiens balsamina-derived antimicrobial peptides, Ib-AMP4 and VESCA3.

FIG. 3 shows the hemolytic activity of melittin, Ib-AMP4, and VESCA3.

### MODES OF THE INVENTION

Hereinafter, the present invention will be described in detail.

The present invention provides an Impatiens balsamina-derived peptide consisting of the amino acid sequence of General Formula 1:

(N-terminus)-GPX¹RRYX²RR-(C-terminus) [General Formula 1]

In the above formula, X¹ is any one selected from the group consisting of glycine (G) and tryptophane (W), and X² is any one selected from the group consisting of cysteine (C), alanine (A), and tryptophane (W).

In the present invention, X¹ may be any one selected from the group consisting of G and W, and X² may be any one selected from the group consisting of C, A, and W. Specifically, X¹ may be any one selected from the group consisting of G and W, and X² may be any one selected from the group consisting of C and W.

In the present invention, the peptide may consist of any one amino acid sequence selected from the group consisting of the amino acid sequences of SEQ ID NOs: 3 to 5, and more particularly, may consist of the amino acid sequence of SEQ ID NO: 3 or 5, and even more particularly, may consist of the amino acid sequence of SEQ ID NO: 5.

The term “peptide” used herein refers to a linear molecule formed by linking amino acid residues by peptide bonds. The peptide may be prepared by a chemical synthesis method known in the art.

In addition, the peptide may have an amidated C-terminus.

In the present invention, the Impatiens balsamina-derived peptide has antimicrobial activity, specifically, has antimicrobial activity against the Escherichia genus or the Staphylococcus genus, and more specifically, has antimicrobial activity against a microorganism of the Escherichia genus, such as Escherichia coli or a microorganism of the Staphylococcus genus, such as Staphylococcus aureus.

In addition, the Impatiens balsamina-derived peptide has antifungal activity, specifically, the antifungal activity against the Candida genus or the Aspergillus genus, and more specifically, the antifungal activity against a microorganism of the Candida genus such as Candida albicans or a microorganism of the Aspergillus genus such as Aspergillus niger.

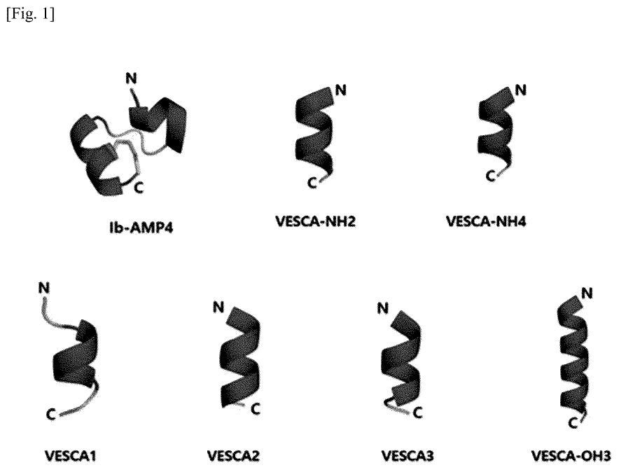

In an exemplary embodiment of the present invention, the present inventors selected six types of peptide derivatives, such as VESCA-NH2 (SEQ ID NO: 1), VESCA-NH4 (SEQ ID NO: 2), VESCA1 (SEQ ID NO: 3), VESCA2 (SEQ ID NO: 4), VESCA3 (SEQ ID NO: 5), and VESCA-OH3 (SEQ ID NO: 6) through 3D structural analysis based on the sequence of Ib-AMP4 (SEQ ID NO: 8), which is reported to have excellent antifungal activity, as an Impatiens balsamina-derived AMP (see Table 1 and FIG. 1).

In addition, the present inventors analyzed the antimicrobial and antifungal activity of the six types of peptide derivatives and Ib-AMP4 against three types of bacterial strains, such as Escherichia coli, Staphylococcus aureus and Cutibacterium acnes, and two types of fungal strains, such as Candida albicans and Aspergillus niger. As a result, it was confirmed that Ib-AMP4 has insignificant antimicrobial activity against Escherichia coli and Staphylococcus aureus, but VESCA3 has excellent antimicrobial activity. In addition, against Candida albicans and Aspergillus niger, it was confirmed that VESCA1 to VESCA3 have the same or excellent antifungal activity compared to Ib-AMP4, and among these, VESCA3 has the best antifungal activity (see Table 2).

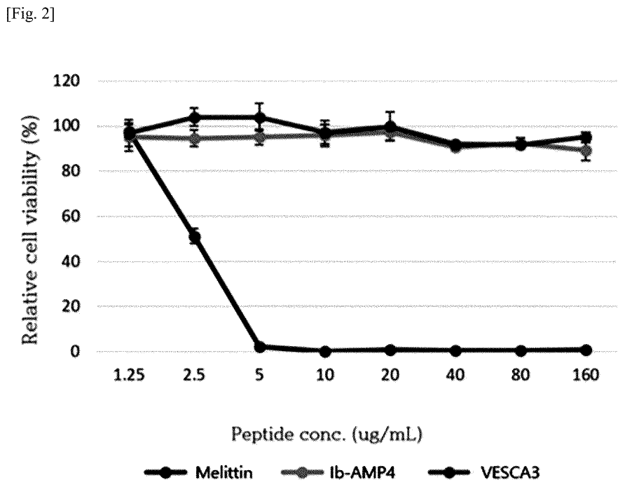

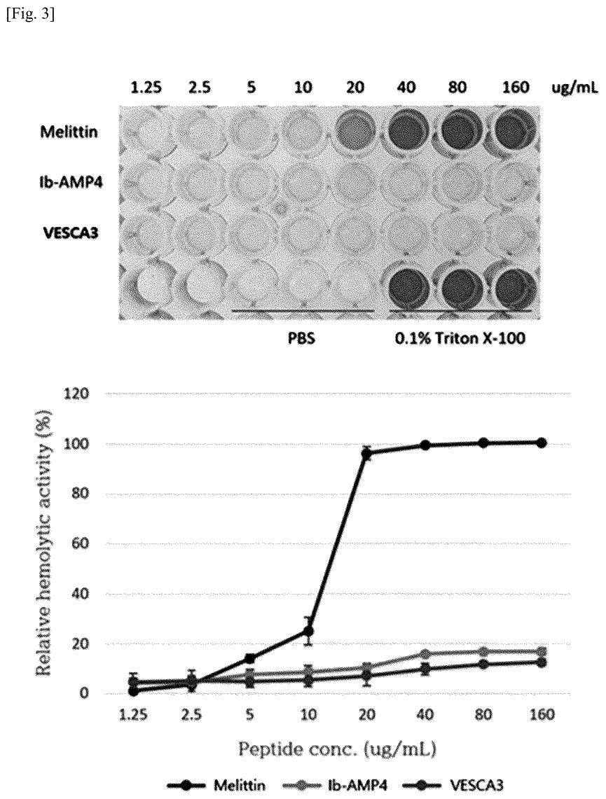

Moreover, as a result of analyzing the cytotoxic and hemolytic activity of VESCA3 that has the best antifungal activity, the present inventors confirmed that it has insignificant cytotoxic and hemolytic activity like Ib-AMP4 (see Tables 3 and 4, and FIGS. 2 and 3).

Therefore, in the present invention, six types of Impatiens balsamina-derived peptide derivatives were prepared, and among these, peptide derivatives having significantly excellent antimicrobial and antifungal activity and insignificant cytotoxic and hemolytic activity compared to Ib-AMP4 were identified. The peptide derivatives are AMPs, which may be used as an antimicrobial or antifungal pharmaceutical composition, antibiotic, food preservative, cosmetic preservative, pharmaceutical preservative, or feed additive, which is used in the pharmaceutical, cosmetic, and food industries.

In addition, the present invention provides an antimicrobial or antifungal composition that includes the Impatiens balsamina-derived peptide as an active ingredient.

In the present invention, the antimicrobial or antifungal composition, as a composition that has the activity of inhibiting the growth of microorganisms such as bacterial strains or fungi, may include all types used in various fields requiring an antimicrobial or antifungal effect. For example, the antimicrobial or antifungal composition may be the form of a drug, a quasi-drug, a food additive, a cosmetic additive, or a feed additive. In addition, it may be used as an antibiotic or preservative in the pharmaceutical field, for preservative or antimicrobial purposes in food, for antimicrobial, sterilizing or disinfecting purposes in agriculture, or for preservative, antibacterial or sterilizing purposes in cosmetics or household items for reducing dandruff, preventing athlete's foot, and reducing underarm odor, or detergents, but the present invention is not limited to the above purposes. Accordingly, the antimicrobial or antifungal composition according to a preparation example of the present invention may be prepared in the form of pharmaceutical composition, such as an antibiotic, a pharmaceutical preservative, a food preservative, a cosmetic preservative, or a feed additive.

The antimicrobial or antifungal composition of the present invention may be an antimicrobial or antifungal composition against, particularly, the Escherichia genus, the Staphylococcus genus, the Candida genus, or the Aspergillus genus, and more particularly, Escherichia coli, Staphylococcus aureus, Candida albicans, or Aspergillus niger, but the present invention is not limited thereto.

Therefore, the present invention provides an antimicrobial or antifungal pharmaceutical composition, which contains the Impatiens balsamina-derived peptide as an active ingredient.

The antimicrobial or antifungal pharmaceutical composition is used for preventing or treating an infectious disease. Specifically, the infection disease is a disease caused by intestinal pathogenic bacteria, and more specifically, Escherichia coli or Staphylococcus aureus, such as food poisoning, typhoid fever, peritonitis, ulcerative colitis, Crohn's disease, intestinal Behcet's disease, infectious diarrhea, gastroenteritis, inflammatory bowel disease, neuropathic enteritis syndrome, small intestinal microbial overgrowth, acute intestinal diarrhea, or ischemic enteritis, but the present invention is not limited thereto. In addition, the infection disease is a disease caused by a fungal strain, specifically, Candida albicans or Aspergillus niger, such as candidiasis, candidal stomatitis, candidal hyperplasia, athlete's food, or aspergillosis, but the present invention is not limited thereto.

The antimicrobial or antifungal pharmaceutical composition of the present invention may be prepared by including one or more pharmaceutically acceptable carriers in addition to the above-described active ingredient. The pharmaceutically acceptable carrier may be used in combination with saline, sterile water, Ringer's solution, buffered saline, dextrose solutions, maltodextrin solutions, glycerol, ethanol, or one or more ingredients thereof, and as needed, may further include another conventional additive such as an antioxidant, a buffer, or a bacteriostatic agent. In addition, the antimicrobial or antifungal pharmaceutical composition of the present invention may be formulated in an injectable form such as an aqueous solution, a suspension, or an emulsion, a pill, a capsule, a granule, or a tablet by additionally adding a diluent, a dispersant, a surfactant, a binder, and a lubricant. Furthermore, the antimicrobial or antifungal pharmaceutical composition of the present invention is preferably prepared according to a disease or ingredient by an appropriate method used in the corresponding field.

The antimicrobial or antifungal pharmaceutical composition according to a preparation example of the present invention may be administered orally or parenterally by a desired method, and the dose may vary according to a subject's body weight, age, sex, health condition, diet, administration time, administration method, excretion rate, and disease severity. The daily dose of the peptide may be 2 to 10 mg/kg, and preferably, 4 to 8 mg/kg, or may be changed according to clinical trial results. The daily dose of the peptide may be administered once or in divided portions a day, but the present invention is not limited thereto.

In addition, the present invention provides an antibiotic, food preservative, cosmetic preservative, or pharmaceutical preservative, which contains the Impatiens balsamina-derived peptide as an active ingredient.

Here, the food preservative, cosmetic preservative, and pharmaceutical preservative may include a disinfectant and an antioxidant as additives that are used to prevent deterioration, decay, discoloration, and chemical changes in food or pharmaceuticals, and further includes a functional antibiotic that inhibits the growth of decaying microorganisms or has a sterilizing effect in food and pharmaceuticals by inhibiting the proliferation of microbes such as bacteria, fungi, and yeast. The ideal conditions for such food preservatives, cosmetic preservatives, and pharmaceutical preservatives are that they are non-toxic and have efficacy even in trace amounts.

The food preservative according to a preparation example of the present invention may be prepared by including one or more known food preservatives. As the food preservative, dehydroaceic acid, potassium sorbate, calcium sorbate, sodium benzoate, calcium benzoate, methyl paraoxybenzoate, propyl paraoxybenzoate, sodium propionate, or calcium propionate is used, but the present invention is not limited thereto.

The food preservative according to a preparation example of the present invention may include 0.01 to 1 wt % of the peptide with respect to the total weight of the food preservative, but the present invention is not limited thereto.

The cosmetic preservative according to a preparation example of the present invention may be prepared by being included in general emulsified and solubilized formulations. Emulsified cosmetics include a nourishing toner, a cream, and an essence, and solubilized cosmetics include a skin toner.

In addition, the cosmetic preservative of the present invention may include a dermatologically acceptable medium or base, in addition to the peptide, to be prepared in the form of an adjuvant for topical or systemic application conventionally used in the field of dermatology. In addition, suitable cosmetic formulations may include, for example, a solution, gel, solid or pasty anhydrous product to which the peptide is added, an emulsion, suspension, microemulsion, microcapsule, microgranule, or ionic type (liposome), obtained by dispersing an oil phase in an aqueous phase, or a non-ionic vesicular dispersion, cream, skin, lotion, powder, ointment, spray, or conceal stick. In addition, the cosmetic preservative of the present invention may be prepared in the form of a foam or an aerosol composition further including a compressed propellant.

In addition, the present invention provides a feed additive including the Impatiens balsamina-derived peptide as an active ingredient.

The feed additive according to a preparation example of the present invention is provided as feed by appropriately mixing it with feed raw materials, and as the feed raw materials, grain oil, chaffs and brans, vegetable oil cake, animal feed raw materials, other feed raw materials, or refined products are used, but the present invention is not limited thereto.

Hereinafter, the present invention will be described in detail with reference to examples and experimental examples.

However, the following examples and experimental examples are merely illustrative of the present invention, and the content of the present invention is not limited by the following examples and experimental examples.

### <Example 1> Preparation of Impatiens balsamina-Derived Antimicrobial Peptide

Ib-AMP (SEQ ID NO: 7) known as an Impatiens balsamina-derived antimicrobial protein (AMP) includes repeated AMP sequences, and four types (Ib-AMP1, Ib-AMP2, Ib-AMP3, and Ib-AMP4) of Ib-AMPs have been reported. Among these, as shown in Table 1 below, six types of peptide derivatives were selected by using Ib-AMP4 (SEQ ID NO: 8), which has been reported to have relatively excellent antifungal activity as a motif.

Specifically, the structural analysis of six types of peptide derivatives was performed based on the Ib-AMP4 sequence using an in silico prediction computational modeling program (PEP-FOLD3), and the peptide derivatives were analyzed and designed and modified based on the formed 3D structures.

Therefore, as shown in FIG. 1, a peptide derivative group (VESCA-NH2, VESCA-NH4) based on the N-terminus helix, a peptide derivative group (VESCA1, VESCA2, and VESCA3) based on the C-terminus helix, and a peptide derivative group in which two helices are combined (VESCA-OH3) were selected, and the selected six types of peptide derivatives were synthesized.

TABLE 1

| | Molecular weight (Da) | | |
|---|---|---|---|
| Name | Theoretical | observed | SEQ ID NO: |
| Ib-AMP4 | 2542.96 | 2543.14 | 8 |
| VESCA-NH2 | 1080.21 | 1080.80 | 1 |
| VESCA-NH4 | 1270.53 | 1271.02 | 2 |
| VESCA1 | 1119.31 | 1120.09 | 3 |
| VESCA2 | 1087.24 | 1088.08 | 4 |
| VESCA3 | 1331.53 | 1332.32 | 5 |
| VESCA4 | 2142.53 | 2142.76 | 6 |

* The peptides have an amidated C-terminus.

### <Experimental Example 1> Confirmation of Antimicrobial and Antifungal Activity of Impatiens balsamina-Derived AMPs

To examine the antimicrobial and antifungal activity of the six types of peptide derivatives prepared in Example 1, antimicrobial and antifungal activity was analyzed using three types of bacterial strains and two types of fungal strains.

Specifically, to analyze antimicrobial and antifungal activity, a minimal inhibitory concentration (MIC) was measured. The three types of bacterial strains included Escherichia coli KCCM 41290, Staphylococcus aureus KCTC 1621, and Cutibacterium acnes KCTC 3314, and the two types of fungal strains included Candida albicans KCTC 17712, and Aspergillus niger KCTC 6317. The culture of Escherichia coli and Staphylococcus aureus was performed in Luria-Bertani (LB) medium (BD Difco), and the culture of Cutibacterium acnes was performed in reinforced clostridial medium (RCM, BD Difco). The culture of Candida albicans and Aspergillus niger was performed in potato dextrose (PD) medium (BD Difco). The pre-cultured microorganisms or spores were diluted in a medium to 5×10³ CFU/mL, and each of the six types of peptide derivatives prepared in Example 1 was added at a predetermined concentration. Afterward, a bacterial strain was cultured at 37° C., a fungal strain was cultured at 25° C. for 18 to 24 hours, and optical densities (OD₆₀₀) were measured using a microplate reader. Fluorescence values (Ex₅₆₀ and Em₆₀₀), instead of optical densities, were measured after adding a resazurin reagent to the Aspergillus niger medium to have a concentration of 100 μM. As a control, Ib-AMP4 was used, and the analysis results were expressed as the average of three repeated experiments.

As a result, as shown in Table 1, against the representative gram-negative bacteria, E. coli, Ib-AMP4 showed very low activity (>100 μg/mL), meanwhile, VESCA3 and VESCA-OH3 had MICs of 60 μg/mL and 40 μg/mL, respectively, indicating very excellent antimicrobial activity.

The result against the representative gram-positive bacteria Staphylococcus aureus was similar to that against E. coli. Compared with Ib-AMP4 (>100 μg/mL), both VESCA3 and VESCA-OH3 showed a high antimicrobial activity of 5 g/mL.

In the case of Candida albicans of the two types of fungal strains, VESCA1 to VESCA3 showed antifungal activity, which is the same as or greater than the control Ib-AMP4 (60 μg/mL). Particularly, VESCA3 has an MIC (20 μg/mL) three-fold lower than the control Ib-AMP4, exhibiting the highest antifungal activity. In the case of Aspergillus niger, VESCA1 to VESCA3 and VESCA-OH3 showed antifungal activity which is the same as or greater than Ib-AMP4 (80 μg/mL). Particularly, VESCA3 has a MIC (40 μg/mL) two-fold lower than Ib-AMP4 (80 μg/mL), exhibiting the highest antifungal activity.

TABLE 2

| | MIC (μg/mL) | | | | | | |
|---|---|---|---|---|---|---|---|
| | Ib- | VESCA- | VESCA- | | | | VESCA- |
| Strain | AMP4 | NH2 | NH4 | VESCA1 | VESCA2 | VESCA3 | OH3 |

Bacteria

| | | | | | | | |
|---|---|---|---|---|---|---|---|
| Escherichia coli | >100 | >100 | >100 | >100 | >100 | 60 | 40 |
| Staphylococcus | >100 | >100 | >100 | >100 | >100 | 5 | 5 |
| aureus | | | | | | | |
| Cutibacterium acnes | >100 | >100 | >100 | >100 | >100 | >100 | >100 |

Fungi

| | | | | | | | |
|---|---|---|---|---|---|---|---|
| Candida albicans | 60 | >100 | 100 | 40 | 60 | 20 | 100 |
| Aspergillus niger | 80 | >100 | >100 | 60 | 80 | 40 | 80 |

### <Experimental Example 2> Confirmation of Cytotoxicity of Impatiens balsamina-Derived AMPs

Cytotoxicity to dermal cells was analyzed using VESCA3 with the best antifungal activity in Experimental Example 1.

Specifically, cytotoxicity was evaluated using a skin cell line, L929 cells (murine fibroblast cell line). In addition, as comparative groups, Ib-AMP4 and the bee venom-derived AMP, melittin, known to be highly cytotoxic, were used. The L929 cells were cultured in RPMI 1640 medium (Gibco), which was supplemented with 10% FBS (Sigma), antibiotic-antimycotic cocktail, penicillin G (100 IU/mL), streptomycin (100 μg/mL), and amphotericin B (250 ng/ml; Gibco), at 37° C. in 5% CO₂. The cells were seeded in a 96-well plate at 1×10⁴ cell/well, incubated for 18 hours, treated with each peptide at 1.25 to 160 g/mL, and then incubated for 24 hours. 10 μL of WST-1 reagent was added to 100 μL of the medium and allowed to react at 37° C. for 30 minutes, and then absorbance (A₄₅₀) was measured at 450 nm using a microplate reader. Instead of the peptides, 0.1% Triton X-100 and PBS were used as positive and negative controls, respectively, and cell viability was calculated based on the following equation: % Cell viability=[(A₄₅₀ in peptide solution−A₄₅₀ in PBS)/(A₄₅₀ in 0.1% Triton X−100-A₄₅₀ in PBS)]×100. The analysis results were expressed as mean±S.D of three repeated experiments.

As a result, as shown in Table 3 and FIG. 2, melittin showed a cell viability of 2.1% at a very low concentration of 5 μg/mL, whereas Ib-AMP4 and VESCA3 showed similar patterns and a cell viability of 90% or more even at 160 g/mL.

TABLE 3

| Concentration | | Relative cell viability (%) | | | | | | | |
|---|---|---|---|---|---|---|---|---|---|
| (μg/mL) of peptide | | 1.25 | 2.5 | 5.0 | 10 | 20 | 40 | 80 | 160 |
| Melittin | Mean | 97.4 | 51.1 | 2.1 | 0.2 | 0.8 | 0.3 | 0.5 | 0.6 |
| | (S.D) | (3.01) | (3.23) | (0.86) | (0.22) | (0.78) | (0.57) | (0.75) | (0.57) |
| Ib-AMP4 | Mean | 95.0 | 94.4 | 95.1 | 95.7 | 97.3 | 90.4 | 92.2 | 89.2 |
| | (S.D) | (6.16) | (3.62) | (3.44) | (4.92) | (3.56) | (1.34) | (2.58) | (4.69) |
| VESCA3 | Mean | 96.7 | 103.8 | 103.8 | 97.1 | 99.7 | 91.9 | 91.6 | 94.9 |
| | (S.D) | (5.85) | (3.92) | (5.95) | (5.24) | (6.51) | (0.57) | (0.22) | (2.30) |

### <Experimental Example 3> Confirmation of Hemolytic Activity of Impatiens balsamina-Derived AMPs

Hemolytic activity was analyzed using VESCA3 with the best antifungal activity in Experimental Example 1.

Specifically, hemolytic activity was analyzed using sheep red blood cells (sRBCs). In addition, as comparative groups, Ib-AMP4 and melittin were used. Defibrinated sheep blood (MB cells) was centrifuged (4,000×g, 10 min, 4° C.), washed with PBS three times, and suspended in PBS, thereby preparing an sRBC solution (10% sRBC). 5% sRBC and each peptide were mixed to have a final concentration of 1.25 to 160 μg/mL, allowed to react at room temperature for 30 minutes, and centrifuged to collect a supernatant. The absorbance (A₄₁₄) of hemoglobin eluted from the red blood cells in the collected supernatant was measured at 414 nm. 0.1% Triton X−100 was used as a positive control, PBS was used as a negative control, and hemolytic activity (% Hemolysis) was calculated based on the following equation: % Hemolysis=[(A₄₁₄ in peptide solution−A₄₁₄ in PBS)/(A₄₁₄ in 0.1% Triton X−100−A₄₁₄ in PBS)]×100.

As a result, as shown in Table 4 and FIG. 3, melittin showed a high hemolytic activity of close to 100% at a very low concentration of 20 μg/mL, whereas Ib-AMP4 and VESCA3 showed a low hemolytic activity of 20% or less even at a high concentration of 160 μg/mL.

TABLE 4

| Concentration | | Hemolytic Activity (%) | | | | | | | |
|---|---|---|---|---|---|---|---|---|---|
| (μg/mL) of peptide | | 1.25 | 2.5 | 5.0 | 10 | 20 | 40 | 80 | 160 |
| Melittin | Mean | 0.98 | 3.40 | 14.0 | 25.02 | 96.10 | 99.36 | 100.2 | 100.0 |
| | (S.D) | (3.46) | (3.60) | (1.57) | (5.56) | (2.69) | (0.44) | (0.81) | (0.60) |
| Ib-AMP4 | Mean | 4.09 | 4.47 | 7.42 | 8.42 | 10.15 | 15.79 | 16.61 | 16.75 |
| | (S.D) | (2.02) | (2.50) | (2.07) | (2.71) | (1.84) | (0.87) | (1.21) | (1.36) |
| VESCA3 | Mean | 4.41 | 5.04 | 4.69 | 5.21 | 6.93 | 9.73 | 11.62 | 12.33 |
| | (S.D) | (3.67) | (4.17) | (2.30) | (2.42) | (3.99) | (2.33) | (0.67) | (1.39) |

From the above results, since it was confirmed that Impatiens balsamina-derived peptide derivatives exhibit excellent antimicrobial and antifungal activity and have insignificant cytotoxic and hemolytic activity, it can be seen that the peptide derivatives can be used for safe antibiotics, food preservatives, cosmetic preservatives, pharmaceutical preservatives, and feed additives.

Embodiments for carrying out the invention have been described together with the above best form for carrying out the invention.

### INDUSTRIAL APPLICABILITY

The present invention can be used in industrial fields associated with antimicrobial compositions.

1. An Impatiens balsamina-derived peptide consisting of the amino acid sequence of General Formula 1:
(N-terminus)-GPX¹RRYX²RR-(C-terminus) [General Formula 1]
wherein X′ is any one selected from the group consisting of glycine (G) and tryptophane (W), and X² is any one selected from the group consisting of cysteine (C), alanine (A), and tryptophane (W).

2. The peptide of claim 1, wherein X² is any one selected from the group consisting of C and W.

3. The peptide of claim 1, wherein the peptide consists of any one amino acid sequence selected from the group consisting of the amino acid sequences of SEQ ID NOs: 3 to 5.

4. The peptide of claim 3, wherein the peptide consists of an amino acid sequence of SEQ ID NO: 3 or 5.

5. The peptide of claim 1, wherein the peptide has antimicrobial and antifungal activity.

6. The peptide of claim 5, wherein the peptide has antimicrobial activity against the Escherichia genus or the Staphylococcus genus.

7. The peptide of claim 6, wherein a microorganism of the Escherichia genus is Escherichia coli, and a microorganism of the Staphylococcus genus is Staphylococcus aureus.

8. The peptide of claim 5, wherein the peptide has antifungal activity against the Candida genus or the Aspergillus genus.

9. The peptide of claim 8, wherein a microorganism of the Candida genus is Candida albicans, and a microorganism of the Aspergillus genus is Aspergillus niger.

10. The peptide of claim 1, wherein the peptide has an amidated C-terminus.

11. An antimicrobial or antifungal pharmaceutical composition, comprising the peptide of claim 1 as an active ingredient.

12. An antibiotic comprising the peptide of claim 1 as an active ingredient.

13. A food preservative comprising the peptide of claim 1 as an active ingredient.

14. A cosmetic preservative comprising the peptide of claim 1 as an active ingredient.

15. A pharmaceutical preservative comprising the peptide of claim 1 as an active ingredient.

16. A feed additive comprising the peptide of claim 1 as an active ingredient.

17. A method of providing at least one of antimicrobial activity and antifungal activity, the method comprising administering the peptide of claim 1 to a subject in need thereof.

18. The method according to claim 17, wherein the subject requires antimicrobial activity against the Escherichia genus or the Staphylococcus genus.

19. The method according to claim 17, wherein the subject requires antifungal activity against the Candida genus or the Aspergillus genus.


SEQUENCE LISTING (8 sequences)

SEQ ID NO: 1 - protein, 9 aa, Artificial Sequence
  VESCA-NH2
DEWGRRCCG

SEQ ID NO: 2 - protein, 9 aa, Artificial Sequence
  VESCA-NH4
YEWKRRCCK

SEQ ID NO: 3 - protein, 9 aa, Artificial Sequence
  VESCA1
GPGRRYCRR

SEQ ID NO: 4 - protein, 9 aa, Artificial Sequence
  VESCA2
GPGRRYARR

SEQ ID NO: 5 - protein, 9 aa, Artificial Sequence
  VESCA3
GPWRRYWRR

SEQ ID NO: 6 - protein, 16 aa, Artificial Sequence
  VESCA4
EWVRRKMKFINSLYSW

SEQ ID NO: 7 - protein, 333 aa, Artificial Sequence
  Ib-AMP
MVQKGVVFGVLLILFICSTLTSADSKPNPTKEEEPAKKPDEVSVKSGGPEVSEDQYRHRCCAWGPGRKYCKRWCANAEEAAAAIPEASEELAQEEAPVYSEDQWGRRCCGWGPGRRYCVRWCQNAEEAAAAIPEATEKAQEAPVYSEDQWGRRCCGWGPGRRYCVRWCQNAEEAAAAVAIPEASEKAQEGPVYSEDQWGRRCCGWGPGRRYCVRWCSNAADEVATPEDVEPGQYGRRCCNWGPGRRYCKRWCHNAAEEATLKAFEEEAAREQPVYSEDQWGRRCCGWGPGRRYCRRWCQSAEEAAAFQAGEVTASLMLIMFKACPCMGPVPSV

SEQ ID NO: 8 - protein, 20 aa, Artificial Sequence
  Ib-AMP4
EWGRRCCGWGPGRRYCRRWC